(19) United States
(12) Patent Application Publication
(10) Pub. No.: US 2021/0140931 A1
(43) Pub. Date: May 13, 2021

(54) Method and Device For Measuring Nitrogen Oxides

(71) Applicant: CPK Automotive GMBH & Co. KG, Muenster (DE)

(72) Inventors: Ralf Moos, Bayreuth (DE); Gunter Hagen, Schwarzenbach am Wald (DE); Jaroslaw Kita, Gefrees (DE); Julia Lattus, Bayreuth (DE); Dirk Bleicker, Warendorf (DE); Frank Noack, Ennigerloh (DE); Julia Wohlrab, Bayreuth (DE)

(21) Appl. No.: 17/133,695

(22) Filed: Dec. 24, 2020

Related U.S. Application Data
(63) Continuation of application No. PCT/EP2019/067243, filed on Jun. 27, 2019.

(30) Foreign Application Priority Data
Jun. 28, 2018 (DE) 102018115623.5

(51) Int. Cl. G01N 33/00; G01N 27/12; G01N 27/407

(52) U.S. Cl. CPC G01N 33/0037; G01N 27/4074; G01N 27/125

(57) ABSTRACT

A method and a device for measuring nitrogen oxides in a gas stream. A functional layer in the sensor senses nitrogen oxides and a measurable physical variable of the functional layer changes in direct correlation with the concentration of nitrogen oxide molecules in the gas. The functional layer is made of a material that, when heated to a specific operating temperature and held at that temperature, maintains an equilibrium between storage and desorption of the nitrogen oxide molecules, and this eliminates the need for a regeneration phase, as is the case with a dosimeter. At the specified operating temperature, the gas sensor indicates a direct relationship between the measured variable and the concentration of the surrounding gas.

### BACKGROUND INFORMATION

### Field of the Invention

The invention relates to a method and a device for measuring nitrogen oxides in a gas stream.

### Discussion of Prior Art

Exhaust-gas aftertreatment systems in internal combustion engines are necessary in order to adhere to restrictions on exhaust emissions that are prescribed by the legal regulations. To this end, gas sensors are incorporated into such aftertreatment systems, to ensure efficient and regulated operation of such aftertreatment systems. Many jurisdictions also require on-board diagnosis (OBD) of such systems and these gas sensors are also used to ensure a continuous OBD.

The denitrification of the exhaust gas plays an important role in the field of lean-burn diesel or fuel injected spark ignition engines. When NOx storage catalysts are used, engine-out nitrogen oxides are first stored in the catalyst coating by means of a special storage material. Regeneration phases are initiated from time to time in order to release the stored nitrogen oxides. The reduction exhaust gas atmosphere that then prevails results in the conversion of NOx. NOx sensors are integrated into the system to achieve substantial optimization in exhaust gas cleaning and fuel consumption. For catalysts which convert NOx by means of the so-called selective catalytic reduction (SCR), the reducing agent must be provided separately in the form of ammonia (NH3). NH3 is obtained in situ from a urea-water solution that is metered into the exhaust gas and is known in the automotive field under the tradename “AdBlue”. In order to optimize the consumption of the reducing agent and, at the same time, the NOx conversion, it is important to know the concentration of nitrogen oxide in the exhaust gas.

The measurement range for nitrogen monoxide (NO) in untreated exhaust gas lies between approximately 100-2000 ppm and for nitrogen dioxide (NO2) between 20-200 ppm at an oxygen concentration (O2) between 1 and 15%. The NOx concentration downstream of a catalyst is lower by a factor of one to two decades and thus, it is difficult to measure the NOx concentration downstream of a catalyst (for example, in order to detect a breakthrough) due to the much lower concentrations.

Designing a successful NOx sensor is made all the more difficult by the parameters for selectivity, sensitivity, stability in the exhaust gas, re-producibility, reaction time, detection limit, and, of course, by the cost-planning for large-scale production and use later.

The high temperatures that arise in the combustion processes require that only temperature-stable materials be used in the exhaust system. The high velocities of the exhaust and, particularly, the rapid changes in composition due to the highly dynamic operating modes of a motor vehicle can also lead to temperature fluctuations of the sensor, which may then influence the sensor response signal. The chemical resistance of the materials used also has to be taken into account. Soot particles in the exhaust gas may be deposited on the surface of the sensor elements and inhibit the diffusion of the analyte to the active sensor layer.

A major problem in measuring nitrogen oxides in exhaust gas is a simultaneous reaction of the sensor to other components in the exhaust gas. This reaction is referred to as sensor cross-sensitivity. Cross-sensitivities lead to false interpretations of the measurement signals and, consequently, to incorrect nitrogen oxide measurement values. Thus, cross-sensitivities can prevent optimum operation of an exhaust gas aftertreatment system and result in a decrease in regeneration intervals, which results in increased fuel consumption in the NOx storage catalytic converter and in an increase in the reducing agent consumption in the SCR system. A cross-sensitivity to NH3 occurs in many nitrogen oxide sensors, because there is an additive effect, such as the following reaction to form nitrogen monoxide and water (H20):

2NH3+5/2O2□2NO+3H2O

The NO produced in the NH3 oxidation according to this equation is additively measured.

But it is precisely with the SCR system, which is frequently used in the automotive field and which uses ammonia as a reducing agent, that it is only conditionally possible to distinguish the NOx content from the added NH3. It is possible to determine the untreated NOx emissions and the actual amount of ammonia that is added by incorporating two sensors into the system—one in front of and the other behind the point where the ammonia is added. An additional sensor would then have to be used downstream of the SCR catalytic converter in order to determine its conversion. It is possible that there is an NH3 cross-sensitivity here, too, and that would have to be estimated via models in the engine control unit. It becomes clear that the use of an exhaust-gas aftertreatment system that uses a multiplicity of sensors in order to achieve a minimum on ammonia cross-sensitivity has distinct economic disadvantages.

The existing state-of-the-art gas sensors can be classified according to the electric variable to be measured, for example, as conductometric, amperometric or potentiometric gas sensors.

U.S. Pat. No. 4,770,760 A discloses a multi-stage NOx sensor with a complex ceramic multi-layer structure based on ZrO2 and which is used in the automotive field in various diesel vehicles. The ZrO2 multi-layer structure results in a sensor that is cost-intensive. Furthermore, this sensor has a cross-sensitivity to various gases and a high cross-sensitivity to NH3, and as a result, its suitability in the SCR system is limited.

DE 10 201 2 206 788 A1 discloses a NOx sensor that is designed as a dosimeter. Dosimeters are suitable for measuring low concentrations of analyte. They accumulate the analyte molecules in a sensitive material, which results in a change in the properties of the material, and that results in a change in a measurable physical variable, such as, for example, the electrical resistance of the sensitive material. This sensitive material is provided as a functional layer on an electrode structure. The accumulation of molecules of the analyte in the sensitive material eventually results in a saturation of the material, so that a regeneration phase, i.e., a cleaning phase during which the gas molecules are removed, is required. Thus, the dosimeter operates in a discontinuous manner.

DE 10 2012 010 423 A1 discloses a cylindrical device in multilayer technology as a platform for high-temperature gas detection. This device can be operated as a dosimeter that is thermally regenerated at regular intervals. The sensor response of a semiconductor sensor can, however, also be used at elevated temperatures, for example, at a temperature of 650° C., in order to enable a measurement of the NO concentration, because, at this temperature, NO is only deposited on the surface of the material, without accumulating as in the dosimeter operation.

DE 11 2009 003 552 T5 discloses NOx storage materials that have an electrical property that changes as a function of the amount of NOx loading, and, thus, can be also used as a dosimeter.

### BRIEF SUMMARY OF THE INVENTION

It is an object of the invention to disclose a method for measuring nitrogen oxides and a nitrogen oxide sensor for implementing the method. The method according to the invention is economical in its implementation. The sensor according to the invention has a low ammonia cross-sensitivity.

The sensor according to the invention has a structure similar to the dosimeter mentioned above, but is operated at a higher operating temperature than the dosimeter, with the surprising result that the sensor no longer operates like a dosimeter with discontinuous operation, but as a gas sensor in continuous operation, as will be explained in more detail below. The material used to create a nitrogen oxide sensing layer, i.e., the functional layer, on the sensor is KMnO4/AlO3. Initial experiments have shown that this functional material is surprisingly well suited in a particular way for this mode of operation of the sensor at the higher operating temperature of 600° C. or more.

The sensor has an insulating ceramic substrate, such as, for example, Al2O3. Electrodes made of a precious metal alloy that can withstand the intended high operating temperature, such as a gold (Au) or a platinum (Pt) alloy, are provided on the ceramic substrate, spaced apart from one another. Platinum electrodes were used in several of the sensors used in the initial experiments. The electrodes are separated from one another and are printed directly onto the ceramic substrate, for example, printed as thick-film electrodes using a screen printing process, or as thin-film electrodes, using a thin-film process such as, for example, sputtering or vapor deposition. The electrodes may be arranged in an interdigital embodiment, that is to say, with fingers that extend between each other, in a comb-like manner. An increase in the number of fingers or the spacing on the same surface, i.e., an increase in the “integration density”, depending on the production method or the technology used to create the layer, results in an increase in the empty capacity or a decrease in the measuring resistance due to a parallel connection.

The sensing or functional material of the functional layer is a material that stores NOx at comparatively low temperatures, similar to the aforementioned and previously publicly disclosed “dosimeter”. The functional material is preferably applied as a coating to the electrode structure, for example, in a thick-film screen printing process. The functional material covers the electrodes evenly, in a manner which allows the electrical properties of the layer to be measured. Suitable measurement methods include, for example, impedance measurement at frequencies that range from 3 MHz to 1 Hz. The electric field between the individual fingers of the flat electrode structure extends through both the functional layer and the substrate, whereby the latter, an insulator, does not contribute to the measurement signal.

As mentioned above, the functional layer of the sensor according to the invention is made of potassium permanganate (KMnO4) and aluminum oxide (Al203). In first experiments, a powder was produced by dry impregnation of Al2O3 with an aqueous KMnO4 solution. This powder was calcined at 500° C. and was then able to be processed to a screen-printing paste, using simple, familiar methods. The calcined powder with ethyl cellulose terpineol in a mixing ratio of 1:11 was passed through a three-roll mill several times and so mixed to form a paste suitable for screen printing. After the screen printing process, the functional layer was first dried at 120° C. and then sintered.

The thickness of the layer was approximately 30-60 μm. It is, of course, possible to use other thicknesses to vary the measuring range of the sensor. Tests have shown that there is a direct relationship between the layer thickness and the basic resistance of the sensor layer and its sensitivity. The material thus obtained is porous, which ensures rapid ingress of the gas to the reactive centers that constitute the sensor effect.

A heating element mounted on the underside of the substrate makes it possible to set a constant operating temperature of the sensor. The heating element may also be applied directly onto the ceramic substrate, for example, with thick-film technology and using a screen printing process to print the heating element onto the substrate. In one embodiment of the invention, the heating element layout is a snaking Pt conductor track. An additional voltage tap may be provided in the heated or hot zone, which allows the four-wire resistance to be measured during operation and the measured value used for subsequent temperature adjustment so as to hold the operating temperature as constant as possible.

The layout of the Pt conductor track is adapted to the respective construction of the sensor, so that, together with the sensor geometry and appropriate heat loss mechanisms, the layout maintains homogeneous temperature distribution on the upper side of the sensor where the functional layer is located. The set temperature indicates the operating temperature of the sensor. The electrical properties of the functional layer depend to a great extent on this temperature.

Alternatively, a thermocouple may be provided on the NOx sensor, and which is, for example, printed onto the ceramic substrate in a screen printing process. In this embodiment, too, the ceramic substrate may be an aluminum oxide substrate and the thermoelement may also be printed in a screen printing process. The thermocouple, separated by a layer of insulation, is located practically directly beneath the electrodes and the functional layer, whereby in this embodiment, too, the electrodes may also be constructed as interdigital electrodes. This embodiment with the thermocouple offers the advantage that heating may be regulated directly on the thermocouple, which, due to spatial proximity, effectively measures the temperature of the functional layer. Heat losses, such as those that occur across the thickness of the substrate, do not play any role in this type of heating control, and the temperature of the functional layer is very precisely adjustable.

The sensor according to the invention may be selectively operated either as a dosimeter or as a gas sensor. Continuous operation of the sensor may be desired or required, instead of the discontinuous dosimeter operation with its regeneration phases. Continuous operation is required, when used to purify exhaust from internal combustion engines. The desired mode of operation is selected by selecting the corresponding operating temperature.

To set the sensor for dosimeter operation, operating temperatures are set in the range from about 300° C. to 400° C. These temperatures are relatively low compared to the exhaust gas temperatures of internal combustion engines. In dosimeter mode, the functional material “collects nitrogen oxides”, as explained at the beginning, i.e., the nitrogen oxides are adsorbed and chemically bound in the functional material. Here, practically every incoming NO or NO2 molecule is captured in the functional material. This leads to a change in the electrical properties of the functional material. Once the functional material is completely loaded with the NO or NO2 molecules, the storage capacity of the material is exhausted and no further storage of nitrogen oxides can take place. At this point, no further change in the electrical properties occurs and the sensor must be regenerated. Hence, dosimeter operation is inherently a discontinuous operation. Increasing the temperature results in desorption of the nitrogen oxides, so that the functional material resumes its original state. After the material is cooled back down to the low operating temperature, the original characteristic storage properties of the sensor return.

The sensor according to the invention, i.e., the same sensor just described above as a dosimeter, may be operated at a higher temperature, namely, at an operating temperature greater than 500° C. For example, the sensor may be operated at an operating temperature of 600° C. or even 700° C. First experiments have shown sensor good results at an operating temperature of 600° C. to 650° C. Due to the comparatively higher operating temperature, nitrogen oxides do not accumulate on the functional layer and, thus, a regeneration phase is not required and continuous operation is possible. An equilibrium between storage and desorption of the nitrogen oxide molecules is achieved. The sensor now exhibits what is referred to as a gas sensor response which, in contrast to the dosimeter response, shows a direct dependence of the measured variable from the surrounding gas concentration.

It is particularly advantageous that the initially achieved, comparatively high operating temperature is kept constant in order to maintain the mentioned adsorption and desorption equilibrium and to enable a measurement that is simple to carry out and doesn't require correction factors for different operating temperatures.

A change in the NOx concentration causes a change in the electrical properties of the functional layer, and this change is reflected in a change in impedance or the change in the complex resistance and, thus, is measurable. For this purpose, frequencies f=1 Hz to 3 MHz are suitable, whereby a constant frequency was used in each case in initial, successful experiments.

The sensor constructed according to the invention and the method according to the invention provide the following advantages:

The sensor has either no or low cross-sensitivities to the typical exhaust gas components occurring in the exhaust gas, specifically, the sensor has a lower cross-sensitivity to ammonia (NH3), no cross-sensitivity to H2 or CO, and no reaction with variations in CO2 and H2O.

The sensor may be made in a simple, planar construction in multi-layer technology. This enables a simple and correspondingly economical production, which also enables series or large-scale production.

The materials used for the functional layer are cost-effective.

Selection of material is limited to materials that have already been successfully used in the field of exhaust gas analysis of internal combustion engines. Accordingly, high long-term stability of the sensor can be expected.

The invention relates to a simple/well understood and, therefore well controllable sensor principle. Further developments are possible, for example, with regard to variation of the layer thickness of the electrode material, so as to change the basic resistance or the measuring range.

Expensive materials, such as platinum and lanthanum components, are not required to produce the functional material. Although expensive materials such as platinum or gold are used in the region of the electrodes, the amount of the precious metal needed is relatively low. As a result, it is possible to produce a cost-effective embodiment of the sensor.

Additional research has revealed that the measured NOx value depends on the lambda value (residual oxygen content) in the exhaust gas. It may therefore be advantageous to integrate an O2 measurement into the NOx sensor. This will allow a correction of the measured NOx value in the evaluation electronics, based on the determined oxygen content, and to output a correspondingly corrected NOx value, which can then be taken into consideration in subsequent processes, for example, for exhaust gas aftertreatment.

It is possible to integrate the O2 measurement into the NOx sensor by providing an O2-sensitive layer in addition to the functional layer used for the NOx measurement. This additional O2-sensitive layer may be provided, for example, on the same substrate that carries the functional layer.

Initial experiments have shown that it is advantageous if the O2-sensitive layer contains barium iron tantalate (BFT). This layer may consist essentially of BFT, and more specifically may consist entirely of doped or undoped BFT, because this material exhibits a temperature independence from its characteristic curve for resistance. Within the context of the present invention, temperature-independence refers to the response of the material in the temperature range that is relevant here, i.e., also to a response, which exhibits a temperature-independence from the resistance characteristic only when the temperature is above a limit temperature. For example, this material exhibits a temperature-independent yet oxygen-dependent change in its electrical resistance in a temperature range from 650 to 800° C., and this has proven to be an extremely positive characteristic with regard to integrating an O2 layer based on BFT into the sensor according to the invention. The temperature independence permits a stable signal even under strong fluctuations in the volume of the gas stream. In addition, it has been found that BFT is particularly well suited as a material for the O2-sensitive layer for practical considerations, because it allows the oxygen to be measured in a resistive process. Alternatively or additionally, it is also possible to measure the Seebeck coefficient. This has the advantage that the Seebeck coefficient, i.e., the generation of a voltage difference due to an temperature difference impressed across the material, is independent of the geometry, is, for example, independent of the layer thickness of the O2-sensitive layer. Thus, the variations in the layer thickness that are unavoidable in series production processes do not affect the quality of the measurements and thus the utility of the produced sensors.

If an O2 layer is provided on the NOx sensor and it is intended that the sensor be heated, it may be advantageous to also heat the O2-sensitive layer, so as to maintain it in an optimal temperature range for the measurements or to bring it into this temperature range as quickly as possible after start-up. Therefore, in view of the previously mentioned temperature ranges in which the NOx sensor and the O2 sensor are operated, these temperature ranges being very similar, it may be advantageous to use just a single heating element, such as an electrical resistance heater, to heat both layers to the desired operating temperature and/or to maintain that temperature. This not only simplifies the construction of the inventive sensor, but also simplifies the sensor control, because just a single heating control is needed. The temperature independence of the BFT material supports such a configuration, because the O2-sensitive layer does not require a precisely adjusted temperature that has be maintained within a narrow range and, as a result, the heating control may be constructed primarily to satisfy the requirements of the NOx sensor.

Different sections of the heating element may, however, also provide different heating intensities, i.e., different heating zones. This is achievable even with a single heating element, by providing a corresponding layout of the heating conductor to achieve the desired degree of heating in two or more heating zones that are maintained at different temperatures. Thus, the sensor according to the invention may have a first heating zone for the functional layer and a second heating zone for the O2-sensitive layer.

Depending on the construction or the layout of the heating element, for example, an electrical heating conductor, it is also possible to provide a temperature control that regulates heat only one area of the sensor. For example, a temperature control may be provided only for the area where the nitrogen sensing layer is located or only for the area where the oxygen sensing layer is located. This allows for the simplest possible technical embodiment of the sensor itself and for the control electronics.

In particular, the heating control may be constructed in such a way that it heats the both the NOx and O2 sensing layers as quickly as possible to the desired temperature, yet, in so doing has a heating curve that is flat enough to avoid creating undesirable material stresses in the substrate, which could possibly impair the service life of the sensor.

Classical sintering methods or coating processes, such as screen printing or the like, may be used to apply the O2-sensitive layer to the substrate, such as a ceramic substrate. Aerosol deposition, a process by which the particles are virtually “shot” onto the substrate in a cold state and at a high velocity, is also a suitable coating process to apply the material to the substrate. This process avoids creating the temperature influences that arise in a sintering process and which can have deleterious effects. Moreover, it is possible to achieve very high material densities with the aerosol deposition process.

It is possible to simplify the entire sensor structure by combining the electrical conductors for the individual components. For example, the ground conductors for two individual sensors. i.e., NOx and O2 sensors, may be combined.

A cap may be provided to protect the entire sensor from undesired external influences, preferably a double-walled cap, as will be discussed below. One function of the protective cap is to protect the sensor from mechanical effects during transport, storage and when the sensor is assembled in an exhaust gas line. For example, if condensate is produced in the exhaust tract of an internal combustion engine after the engine has been shut down, this condensate can strike the already heated sensor during the warm-up phase. The cap serves to protect the sensor from the ‘water shock’ that the condensate could cause. There is always a risk that stress cracks occur in the ceramic substrate and the cap shields the sensor from water shock and from the negative temperature peaks associated therewith, that is to say, protects the sensor against sudden cooling.

The cap also protects the sensor from temperature spikes, that is to say, protects the sensor against short-term overheating, which may occur during operation in the exhaust gas flow. Similarly, the cap also protects the sensor against intensive heat radiation, particularly after the engine has been turned off, which could act on the unprotected sensor.

Surprisingly, it has also been found that, more than just providing the protections mentioned above, the cap may also serve to guide the stream of gas along the sensor. This requires a suitable construction of the cap. Specifically, at least one inlet opening and at least one outlet opening are provided in the wall of the cap. These openings on the cap are located such, that an overpressure and an overpressure are generated specific locations on or in the cap, the difference in pressure effectively directing the gas stream along the desired path. Depending on the particular installation or the design of the exhaust system, practical tests will show the optimal placement of the openings with regard to response characteristics on the one hand and the measurement variable on the other.

The cap is preferably double-walled. One benefit of the double-wall construction is that it optimizes the protective effects mentioned above. But it also makes it possible to design a path for the gas flow inside the wall of the cap that results in a particularly uniform stream of gas onto the NOx sensor and the O2 sensor, if one is provided.

Optionally, a catalytic coating may be provided on the cap in order to bring about an additional reaction that works to reduce cross-sensitivities, such as may occur with respect to ammonia (NH3).

Preferably, the sensor has a freely rotatable connector, so that the sensor may positioned and oriented in a freely determinable angular position in the flow path of the exhaust gas. To this end, the sensor may be arranged in a retainer or housing and, together with the holder or housing, be mounted so as to be freely rotatable relative to connector elements. The connector elements to mount the sensor may be constructed as threaded sleeves, mounting flanges, or the like.

The thermal element previously mentioned may be used to regulate the temperature, or, alternatively, a platinum (Pt) temperature sensor.

### BRIEF DESCRIPTION OF THE DRAWINGS

The present invention is described with reference to the accompanying drawings. The drawings are purely schematic representations and like reference numbers indicate identical or functionally similar elements.

FIG. 1 is an exploded view that illustrates the structure of a sensor according to the invention for measuring nitrogen oxides,

FIG. 2 is a cross-sectional view of the sensor.

FIG. 3 is a graph that shows the complex impedances of the sensor with different gas compositions.

FIG. 4 shows the sensor response when measuring a basic gas and with various concentrations of gas that have been introduced.

FIG. 5 illustrates the upper side of a first embodiment of the sensor.

FIG. 6 illustrates the upper side of a second embodiment of the sensor.

FIG. 7 is a plane view of the upper side of the substrate for the first embodiment of the sensor, showing the electrodes and the heating element

FIG. 8 is a plane view of the upper side of the substrate for the second embodiment of the sensor, showing the electrodes and the layout of the heating element.

FIG. 9 is a longitudinal cross-section through an installation-ready assembly that contains the sensor for measuring nitrogen oxides and a cap that also serves to guide the stream of exhaust along a particular path.

### DETAILED DESCRIPTION OF THE INVENTION

The present invention will now be described more fully in detail with reference to the accompanying drawings. This invention should not, however, be construed as limited to the embodiments set forth herein; rather, the drawings are provided so that this disclosure will be complete and will fully convey the scope of the invention to those skilled in the art.

FIG. 1 shows a sensor 1 according to the invention which has a ceramic substrate 2 that is made of aluminum oxide. Two electrodes 3 are printed onto the ceramic substrate 2 in an interdigital arrangement, using a thick-film screen printing process. The electrodes 3 are completely covered by a functional layer 4, which is made of a material combination of potassium permanganate and aluminum oxide. Also provided on the ceramic substrate 2 is a temperature sensor 6, which, in the embodiment shown, is a thermocouple.

FIG. 2 is a cross-sectional view of the sensor 1, showing a heating element 5 that is printed onto the underside of the ceramic substrate 2 in a thick-film screen printing process.

FIG. 3 shows a Nyquist plot of the complex impedances of the sensor 1 at an operating temperature of 635° C. for two different gas compositions: the upper curve shows the sensor response, i.e., the measured values obtained from the sensor 1, in a basic gas and the lower curve shows the sensor response in a gas that is otherwise identical to the basic gas but which now contains 400 ppm of nitrogen oxide NO.

FIG. 4 shows two graphs, one above the other. The lower graph shows the ohmic component, calculated over time from the complex impedance of the sensor 1, based on an RIIC parallel circuit. This measurement was carried out at an operating temperature of 600° C. and a frequency of 100 kHz, using the sensor 1 with its functional layer 4 made from potassium permanganate and aluminum oxide.

The upper graph in FIG. 4 shows the composition of the gas, which contains various concentrations of gases added to the basic gas at specific times. A horizontal line in the upper graph at approximately the middle of the graph indicates the basic gas, which contains a concentration of CO2 of approximately 3%. This concentration of CO2 was maintained constant for most of the time of the analysis, with an exception at approximately 40 minutes. This upper graph also shows that the level of oxygen O2 in the gas was held constant at approximately 5%.

The bars in the upper graph at approximately 4 and 11 min indicate a metered addition of nitrogen oxide NO to the basic gas; the lower graph shows that the time-identical sensor responses correlate to the changes in the gas composition.

The next two chronologically following bars in the upper diagram show a metered introduction of carbon monoxide CO at about 15 min and hydrogen H2 at about 22 min. The lower graph shows that there is no sensor response, i.e., the sensor 1 is not sensitive to these gases.

The next two bars indicate a metered introduction of ammonia NH3 at approximately 28 and 35 minutes, and specifically, in different concentrations. The sensor 1 shows a relatively low sensitivity to this gas, as can be seen in the very slight dips at the corresponding times in the lower graph.

The two bars at the right-side end in the upper graph relate to a metered introduction of carbon dioxide CO2 at about 42 min and of water vapor H2O at about 46 min. The lower graph shows that the sensor 1 does not exhibit any cross-sensitivity to these gases.

FIG. 5 shows the previously described first embodiment of the sensor 1, which is designed as an exclusive NOX sensor, and in which the two electrical conductors 3 are provided on the ceramic substrate 2 and are covered in some regions by the functional layer 4.

FIG. 6 shows a second embodiment of the sensor 1, which is a combined NOX and O2 sensor. This combination sensor allows the evaluation electronics to take correction factors into account, based on the detection of the residual oxygen content in the exhaust gas. The measured NOX value is dependent on the lambda value, i.e., the residual oxygen content in the exhaust gas, and this allows the measured NOX value to be corrected, even under conditions with different lambda values, by applying such correction factors. Thus, the actual NOX value can be calculated or displayed or taken into account in the exhaust gas aftertreatment.

In this second embodiment of the sensor 1, an O2-sensitive layer 7 is provided on the ceramic substrate 2 and is connected to two additional electrical conductors 8. As in the first embodiment illustrated in FIG. 5, the electrical conductors 3 terminate at the lower end of the sensor 1 in contacts 9, and the additional conductors 8 also end with similar contacts 9, so that a single connector plug with the corresponding number of electrical connectors is used to connect to the sensor and provide the electrical contacts that are connected to, for example, an electronic read-out unit.

FIG. 7 shows the view of the underside of the first embodiment of the sensor that is shown in FIG. 5. The heating element 5 provided there serves to indirectly heat the functional layer 4, namely, heating the region on the upper side where the functional layer 4 is located is done by heating the underside of the ceramic substrate 2. Electrical contacts 9 are also provided on the underside of the ceramic substrate 2 at the lower end of the ceramic substrate 2, these contacts 9 serving to supply power to the heating element 5.

FIG. 8 shows the underside of the second embodiment of the sensor 1 that is shown in FIG. 6. In this embodiment, the functional layer 4 is also heated by heating the corresponding region of the ceramic substrate 2. This second embodiment of the sensor 1 according to the invention, however, also has an additional heating zone 10, located on the underside in the area that corresponds to the area of the O2-sensitive layer 7 on the upper side of the substrate 2. The heating element 5 is an electrical heating resistor that is printed onto the ceramic substrate 2 in a layout that provides two heating zones. A first zone is indicated by the reference designation 5 and the layout is as a rectangularly running path. A second heating zone 10 is provided at the zigzag-shaped sections of the same electrical conductor.

FIG. 9 illustrates an assembly which includes the sensor 1 as the essential component within a multi-component housing 11, as well as a cap 16. For purposes of clarity, the end of the assembly that contains the electrical contacts 9 is referred to as the rear end and the end with the cap 16 is referred to as the front end. The ceramic substrate 2 shown here is greater in length than the substrate 2 shown in the previously described embodiments. The sensor 1 is held in place in the assembly by means of spring clips 12 that are provided toward the rear end. In the central region of the assembly, a multi-component press-on element 14 serves to hold the sensor 1 in place. The functional layer 4 of the sensor 1 is provided toward the front end of the assembly.

The multi-component housing 11 has a sleeve-like inner body, around which a connector 15 extends circumferentially and which, in the illustrated embodiment is designed as a screw-on sleeve with an external thread. The inner body of the housing 11 is freely rotatable relative to the connector 15. This simplifies the installation of the assembly: the sensor 1 is connected in a rotationally fixed manner to the inner body of the housing 11, and a control device belonging to the sensor 1, along with its cable that runs to the sensor 1, is fixedly connected to the sensor 1. The cable does not get twisted when the screw-on sleeve is rotated relative to the inner body when the sensor assembly is installed.

The front end of the sensor 1 that contains the functional layer 4 is within the cap 16, which, in this embodiment, is a double-walled cap 16. The outer wall of the cap 16 has a plurality of inlet openings 17. The lines with arrows indicate how the gas stream flows through the inlet openings 17 into the gap between the two walls of the cap 16. The gas flows through the gap, parallel to the sensor 1, toward the rear end of the cap 16, where it enters the interior space surrounded by the cap 16, as indicated by the tightly curved arrow lines. These tightly curved arrow lines indicate a reversal in the flow path of the gas, so that the gas, once it enters the interior space of the cap, now streams parallel to the sensor 1 and toward the front end of the cap 16.

An outlet opening 18 is provided at the front end of the cap 16, creating an underpressure which draws the exhaust gas out of the interior of the cap 16. The cap 16 extends forward beyond the front end of the sensor 1 and this results in a uniform flow of the stream of gas across the functional layer 4 and, if present, also across the O2-sensitive layer 7, all the way to their respective front ends. In addition to ensuring an even flow of gas across the relevant sensor elements 4 and 7, the cap 16 also provides optimum protection for the sensor 1 against mechanical and temperature effects, as previously discussed.

In the embodiment shown, the cap 16 is rotationally symmetrical. It is understood that it is also possible to construct the cap 16 so that it is to be assembled in a specific orientation in the gas flow, so as to effect a specific flow onto the sensor 1. In this case, the inner body of the housing 11 may be provided with a marking above the connector 15, so that the desired orientation of the cap is visible from the outside when the assembly is being screwed into the wall of an exhaust gas line. The freely rotatable arrangement of the inner body within the connector 15 makes it easier to maintain the intended alignment of the cap 16 during assembly.

It is understood that the embodiments described herein are merely illustrative of the sensor according to the invention and the method according to the invention of measuring nitrogen oxide in exhaust gas. Variations in the construction of the sensor may be contemplated by one skilled in the art without limiting the intended scope of the invention herein disclosed and as defined by the following claims.

What is claimed is:

1: A device for measuring nitrogen oxide molecules in a stream of gas, the device comprising:
a sensor comprising electrodes and a functional layer for sensing nitrogen oxide, the functional layer made of a material that combines KMnO4 and Al2O3;
wherein nitrogen oxide molecules are adsorbable on the functional layer; and
wherein the sensor has a temperature stability of at least 500° C.

2: The device of claim 1, further comprising:
a ceramic substrate that is electrically insulating;
wherein the electrodes and the functional layer are provided on the ceramic substrate.

3: The device of claim 1, wherein the electrodes are made of a precious metal alloy.

4: The device of claim 3, wherein the precious metal alloy is a gold alloy.

5: The device of claim 3, wherein the precious metal alloy is a platinum alloy.

6: The device of 1, wherein the functional layer is applied as a coating over the electrodes.

7: The device of claim 1, wherein the sensor has an essentially planar, flat structure.

8: The device of claim 1, further comprising:
a heating element that is an electrical resistance heating element.

9: The device of claim 8, wherein the heating element has an electrode that has an additional voltage tap in a heated zone of the sensor,
wherein a four-wire resistance is measurable to obtain a heating value that is used to adjust the temperature on the functional layer.

10: The device of claim 1, further comprising:
a temperature sensor that is provided on the ceramic substrate; and
an insulation layer that is provided over the temperature sensor;
wherein the electrodes and the functional layer are arranged above the insulation layer.

11: The device of claim 10, wherein the temperature sensor is printed onto the ceramic substrate using a screen printing process.

12: The device of claim 1, further comprising:
an O2 sensor that is an O2-sensitive layer that is applied to the ceramic substrate.

13: The device of claim 12, wherein the O2 sensor is a resistive sensor having a temperature-independent characteristic curve.

14: The device of claim 13, the O2 sensor has a substantially temperature-independent but O2-dependent Seebeck coefficient.

15: The device of claim 12, wherein the O2-sensitive layer contains barium iron tantalate (BFT).

16: The device of claim 12, wherein the heating element includes an additional heating zone for heating the O2 sensor.

17: The device of claim 12, wherein the O2-sensitive layer is applied to the ceramic substrate in an aerosol deposition process.

18: The device of claim 1, further comprising:
a cap that surrounds the sensor;
wherein the cap has an inlet opening and an outlet opening so as to guide the stream of gas across the sensor.

19: The device of claim 16, wherein the cap is a double-walled construction.

20: The device of claim 16, wherein the cap has a catalytic coating.

21: The device of 1, further comprising:
a connector element for mounting the sensor in an exhaust system;
wherein the sensor is mounted so as to be freely rotatable relative to the connector element.
